(19) United States
(12) Patent Application Publication
(10) Pub. No.: US 2005/0113742 A1
(43) Pub. Date: May 26, 2005

(54) Portable type irrigator

(76) Inventor: Byung Eun Yoo, Nam-gu (KR)

Correspondence Address: JORDAN AND HAMBURG LLP, 122 EAST 42ND STREET, SUITE 4000, NEW YORK, NY, 10168, US

(21) Appl. No.: 10/816,017

(22) Filed: Mar. 31, 2004

(30) Foreign Application Priority Data
Nov. 20, 2003 (KR) 2003-36260

(51) Int. Cl. A61M 1/00

(52) U.S. Cl. 604/37

(57) ABSTRACT

The portable type irrigator according to the present invention comprises a suction hose, an air pump and a nozzle injection part by which the liquid is passed and the liquid contained in the liquid reservoir is directly injected into a large intestine through the anus or an interior of the vaginal canal to irrigate the large intestine or the vaginal canal by using the supplied liquid, and is characterized in that the suction hose is integrally connected to a flange of the liquid supplying body, the nozzle injection part be connected to a connecting member of the liquid supplying body in a spiral manner, and the air pump is mounted to a lower side of the entrance/exit passage for liquid communicated with a supplying passage of the liquid supplying body.

### BACKGROUND OF THE INVENTION

1. Field of the Invention

The present invention relates to an irrigator for irrigating a large intestine containing coprostasis or an interior of the vaginal canal. More particularly, the present invention relates to a portable type irrigator which can be conveniently and easily used without regard to a place.

2. Description of the Prior Art

As is generally known, the large intestine of man has a function by which moisture is controlled, an electrolyte be maintained, and food residues be made into feces by using a bacteria action. However, if such function of large intestine does not display sufficiently, noxious ingredients and toxins are accumulated in the body so that the large intestine is palsied and old blood vessels are ruptured or expanded. Consequently, the large intestinal diseases such as large intestine cancer, colitis, hypersensitive syndrome, large intestine ulcer and hemorrhoids as well as various kinds of illnesses such as constipation, diarrhea, dyspepsia, thin feces and gas filled in a lower abdominal region are caused by a functional disease of the large intestine and the large intestine becomes a hotbed of bacteria.

Accordingly, since various diseases such as intestine paralysis, functional disease of the intestine and skin senescence are caused by a retention of waste matter in the intestine, in order to eliminate radically such causes, the waste matters in the intestine are eliminated by an irrigation using an intestine irrigator so that the various diseases can be prevented. As described above, by eliminating toxins caused by the coprostasis in the intestine, the intestine irrigator has an excellent effect on prevention and treatment of various diseases.

The conventional intestine irrigator is mainly classified into a mechanical type intestine irrigator and a household intestine irrigator. The former mechanical type intestine irrigator is mainly installed at public institutions such as hospitals or skilled nursing facilities due to its expensive cost and its large size, while the latter household intestine irrigator is widely popularized in small-scale hospitals as well as homes.

The present invention relates to a household intestine irrigator which can be easily and conveniently used by a patient or user. In the structure of such household intestine irrigator, the household intestine irrigator disclosed in the Korean Utility Model Registration No. 218, 980 is a wall mounting type intestine irrigator comprising a liquid reservoir, a plurality of valves, an air pump and various collateral parts. The structure and equipment of the above intestine irrigator are complicated and the intestine irrigator is troublesome in how to use when a patient or user injects the liquid into the large intestine through the anus using an injector, and the installation thereof.

In order to solve the above-mentioned problems, the present inventor has invented a large intestine irrigator which can be more conveniently used than that of Korean Utility Model Application No. 2003-26983 filed by the present inventor.

The large intestine irrigator of the said Korean Utility Model Application No. 2003-26983 proposed by the present inventor has a structure in which an air pump is mounted to a curved pipe connected to a liquid suction hose and a nozzle injection part. Although the above irrigator has an advantage in that the irrigator can be conveniently used, there is an inconvenience in that user grips the air pump to use the irrigator.

Up to now, on the other hand, various irrigators have been proposed for irrigating the interior of women's vaginal canal. Due to complication of the structure and inconvenience of the outer appearance of the irrigator, however, it imposes burden on the user on use. Accordingly, the conventional irrigators have been not widely used.

### SUMMARY OF THE INVENTION

Therefore, an object of the present invention is to overcome the above drawbacks of the prior arts as described above, and to provide an improved portable type irrigator which can be used conveniently and easily for both irrigating a large intestine and an interior of the vaginal canal and has an improved appearance so that the users do not have the burden on use.

The present inventor has extensively studied in order to achieve the above objects. As a result, he has invented a portable type irrigator comprising a suction hose, an air pump and a nozzle injection part by which the liquid contained in the liquid reservoir is injected directly into the interior of the large intestine through the anus or the interior of the vaginal canal, characterized in that the above suction hose is integrally connected to a flange of the liquid supplying body, the above nozzle injection part be connected to a connecting member of the liquid supplying body in a spiral manner, and the air pump be mounted to a lower side of the entrance/exit passage for liquid communicated with a supplying passage of the liquid supplying body.

Other features and objects of the present invention will become more apparent from the following description by referring to the accompanying drawings, and examples only as to how the invention may be put into a practice.

### BRIEF DESCRIPTION OF THE DRAWINGS

The above and other objects, features and advantages of the present invention will become apparent from the following description of preferred embodiments given in conjunction with the accompanying drawings, in which:

FIG. 1 is a perspective view of the portable type irrigator according to the present invention;

FIG. 2 is a sectional view of the operating portable type irrigator of FIG. I;

FIG. 3A and FIG. 3B are views showing the embodiments of a nozzle injection part according to the invention; and

FIG. 4 is a schematic view showing a state where the portable type irrigator according to the present invention is used.

### DETAILED DESCRIPTION OF THE PREFERRED EMBODIMENTS

Hereinafter, embodiments of the present invention will be described in detail by referring to accompanying drawings.

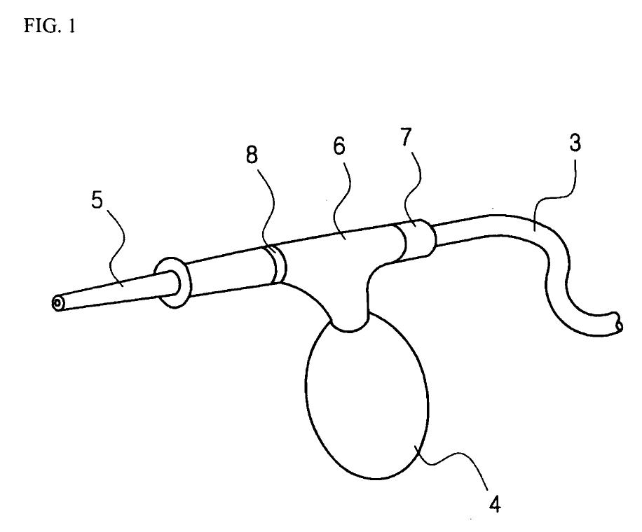

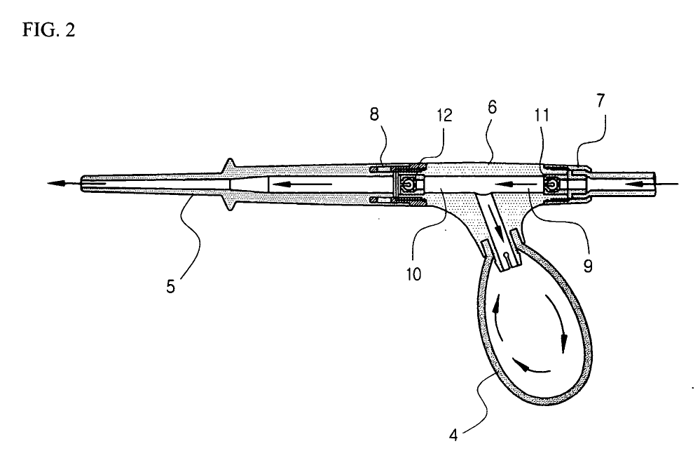

FIG. 1 is a perspective view of the portable type irrigator according to the present invention and FIG. 2 is a sectional view of the operating portable type irrigator of the present invention.

The portable type irrigator according to the present invention comprises a suction hose 3, an air pump 4 and a nozzle injection part 5 by which the liquid 2 contained in the liquid reservoir 1 is injected directly into the interior of the large intestine through the anus or the interior of the vaginal canal, characterized in that the above suction hose 3 is integrally connected to a flange 7 of the liquid supplying body 6, the above nozzle injection part 5 be connected to a connecting member 8 of the above liquid supplying body 6 in a spiral manner, and the above air pump 4 be mounted to a lower side of the entrance/exit passage 10 for liquid 2 communicated with a supplying passage 9 of the above liquid supplying body 6.

That is, since an air pump 4 is attached to the liquid supplying body 6 in the form of handle of shoot gun providing the liquid, the user can grip conveniently and control the air pump 4 so that the irrigator is convenient for use and the user does not have the burden on use.

Also, since the above nozzle injection part 5 is connected to a connecting member 8 of the above liquid supplying body 6 in a spiral manner, when the nozzle injection part 5 to be inserted into an anus is contaminated or stained, the nozzle injection part 5 can be detached and removed, and then can be replaced with the new one so that an effective use can be sought.

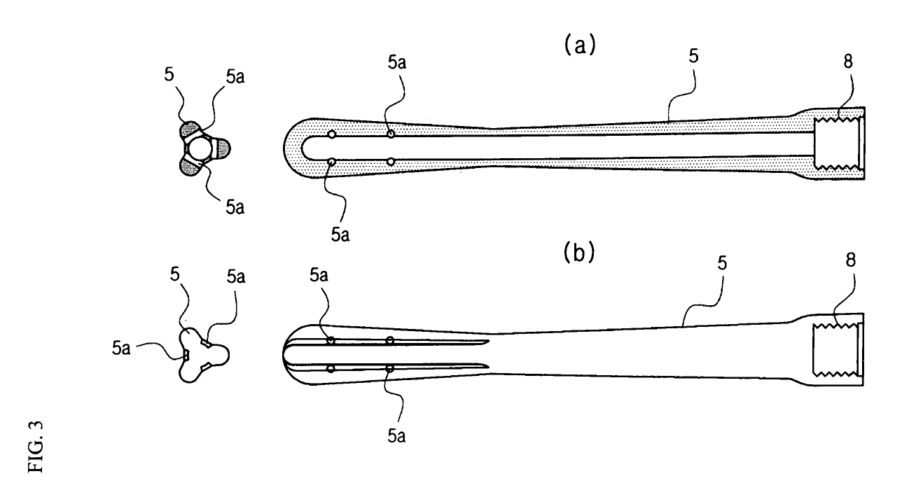

On the other hand, FIG. 3A and FIG. 3B are views showing the embodiments of the nozzle injection part 5 which is used for irrigating the interior of women's vaginal canal. When women's vaginal canal is irrigated, the user may replace only this nozzle injection part with the new one. The above nozzle injection part 5 is mounted to the liquid supplying body 6 by means of a connecting member 8 as done in the previous embodiment. However, on a front end of the nozzle injection part 5, a plurality of holes 5a through which the liquid is supplied are formed so that when the interior of women's vaginal canal is irrigated, the liquid to be supplied into the interior of women's vaginal canal can be effectively ejected.

A method for operating a portable type irrigator according to the present invention set forth hereinafter.

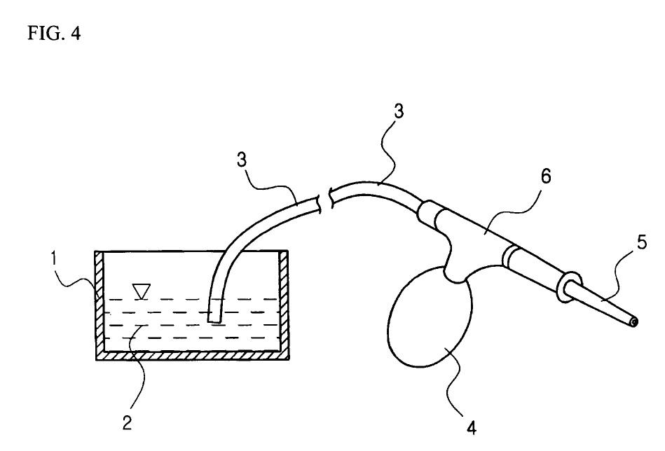

In order to eliminate smoothly the coprostasis from the large intestine or to irrigate the interior of the vaginal canal, the irrigator according to the present invention is constructed to inject the liquid contained in the liquid reservoir 1 into the interior of the large intestine or the interior of the vaginal canal, in which the nozzle injection part 5 is inserted in the anus or the interior of the vaginal canal. In a state where the suction hose 3 is dipped in the liquid 2 of the liquid reservoir 1 as shown in FIG. 4, if an user operates the air pump 4 of the liquid supplying body 6 using a hand, the liquid 2 flows into the air pump 4 in an arrow direction shown in FIG. 2, and then discharged into the nozzle injection part 5. At this time, the first and second check valves 11 and 12 prevent the supplied liquid from flowing backward, and the liquid is discharged into the nozzle injection part 5. Therefore, the liquid is directly injected into the interior of the large intestine through the anus or into the interior of the vaginal canal to irrigate the large intestine or the interior of the vaginal canal.

### Industrial Applicability

The portable type irrigator according to the present invention has an improved irrigator, and may be conveniently used. The present portable type irrigator has an improved appearance and does not have the burden on use. Furthermore, the present portable type irrigator may be conveniently used without regard to a place, and can be used selectively for irrigating the large intestine or the interior of the vaginal canal.

Although the irrigator has been described in connection with the preferred embodiment thereof shown in the accompanying drawings, it is a mere example of the present invention. It can also be understood by those skilled in the art that various changes and modifications thereof can be made thereto without departing from the scope and spirit of the present invention defined by the claims. Therefore, the true scope of the present invention should be defined by the technical spirit of the appended claims.

1. A portable type irrigator comprising a suction hose, an air pump and a nozzle injection part by which the liquid contained in the liquid reservoir is injected directly into the interior of the large intestine through the anus or the interior of the vaginal canal, characterized in that the above suction hose is integrally connected to a flange of the liquid supplying body, the above nozzle injection part be connected to a connecting member of the liquid supplying body in a spiral manner, and the air pump be mounted to a lower side of the entrance/exit passage for liquid communicated with a supplying passage of the liquid supplying body.

2. The portable type irrigator according to claim 1, wherein the above nozzle injection part has a plurality of hole arranged in a radial direction at a front end of the nozzle injection part.
